(19) United States
(12) Patent Application Publication
(10) Pub. No.: US 2004/0018631 A1
(43) Pub. Date: Jan. 29, 2004

(54) Method for detecting fluoride

(76) Inventors: Christopher James Ward, Bath (GB); Prakash Patel, Manchester (GB); Tony David James, Bath (GB)

Correspondence Address: MORGAN LEWIS & BOCKIUS LLP, 1111 PENNSYLVANIA AVENUE NW, WASHINGTON, DC, 20004, US

(21) Appl. No.: 10/381,258

(22) PCT Filed: Sep. 11, 2001
(86) PCT No.: PCT/GB01/04039

(30) Foreign Application Priority Data
Sep. 25, 2000 (GB) 0023439.3

(51) Int. Cl. G01N 33/00

(52) U.S. Cl. 436/125

(57) ABSTRACT

A method for detecting the presence and/or amount of fluoride in a test sample which comprises contacting the test sample with a compound of Formula (1) or a salt thereof and evaluating any change in the spectral characteristics of the compound:

X—[—(CₙH₂ₙ)—NH—(CₘH₂ₘ)—W—B(OH)₂]ₚ Formula (1)

wherein:

X is a chromophore or a fluorophore;

W is an optionally substituted aromatic ring;

n is 0, 1 or 2;

m is 1 or 2; and

p is 1 or 2. Also a kit for detecting the presence and/or amount of fluoride.

The present invention relates to a method for detecting the presence and/or amount of fluoride and to a kit for detecting the presence and/or amount of fluoride.

Fluoride is added to water systems throughout the developed world to arrest the development of tooth decay. To this end it is also added to a range of other dental products such as mouthwash and toothpaste. Fluoride is also becoming a common component of many foodstuffs through the use of fluorinated water in food manufacture. Although low levels of fluoride are known to have beneficial effects in preventing tooth decay, excess levels can cause irreversible damage to the teeth of some individuals. Thus easy to use, cheap and accurate methods of fluoride determination are desirable.

Fluoride can also be harmful to certain fish and many aquarium owners are interested to know the level of fluoride in water they are adding to their fish tanks.

Fluoride concentration is usually determined using a specific electrode. These electrodes are sensitive and selective. However, electrode use is not always convenient especially when a private individual is performing the analysis and so a simple sensitive means of visualisation of fluoride would find considerable application.

Boron centered fluoride receptors have been studied by Katz,(J.Org.Chem. 50, 5027 and J.Am.Chem.Soc, 1986, 108, 7640) who trapped fluoride ions between two electron accepting boron's in 1,8-naphthalendiylbis(dimethylborane). The affect of fluoride on the fluorescence and electrochemistry of aromatic boronic acids was also been investigated by Yuchi (Anal.Chim.Acta.,1999, 387, 189).

The present invention provides a method for detecting the presence and/or amount of fluoride in a test sample which comprises contacting the test sample with a compound of Formula (1) or a salt thereof and evaluating any change in the spectral characteristics of the compound:

X—[—(CₙH₂ₙ)—NH—(CₘH₂ₘ)—W—B(OH)₂]ₚ Formula (1)

wherein:

X is a chromophore or a fluorophore;

W is an optionally substituted aromatic ring;

n is 0, 1 or 2;

m is 1 or 2; and

p is 1 or 2.

Preferred chromophores represented by X are optionally substituted azo (especially monoazo and disazo), anthraquinone, pyrroline, phthalocyanine, polymethine, aryl-carbonium, triphenodioxazine, diarylmethane, triarylmethane, anthraquinone, phthalocyanine, methine, polymethine, indoaniline, indophenol, stilbene, squarilium, aminoketone, xanthene, fluorone, acridene, quinolene, thiazole, azine, nigrosine, oxazine, thiazine, indigoid quinonioid, quinacridone, lactone, pyrroline, benzodifuranone, or indolene group or a combination of such groups.

Preferred fluorophores represented by X are optionally substituted naphthyl, anthryl, pyrenyl, stilbene and phenanthryl or a combination of such groups.

Preferably X is such that when the compound of Formula (1) is dissolved in a solvent the resultant solution is red, orange, yellow, green, blue, indigo or violet.

It is preferred that the compound of Formula (1) is of Formula (2) or a salt thereof:

A—N═N—D—(CₙH₂ₙ)—NH—(CₘH₂ₘ)—E—B(OH)₂ Formula (2)

wherein:

A, D and E are each independently optionally substituted aromatic heterocyclic or homocyclic group and m and n are as hereinbefore defined.

In Formulae (1) and (2) it is preferred that n is 0 and m is 1

Preferred optionally substituted aromatic heterocyclic groups are diazine, thiazole, benzthiazole, benzdiazine, triazole, isoxazole, benzisoxazole, thiadiazole, oxadiazole, isothiazole, benzisothiazole, pyridiazine, triazine, oxazole, thiophene, benzoxazole, pyrimidine or pyridine. Preferred optionally substituted homocyclic groups are optionally substituted phenyl, naphthyl, pyrenyl, stilbenyl.

Preferably A is optionally substituted phenyl and D and E are each independently optionally substituted phenylene.

Preferably the optional substituents which may be present on X, A, D, E and W are each independently selected from alkyl (preferably C₁₋₄-alkyl), alkoxy (preferably C₁₋₄-alkoxy), aryl (preferably phenyl), aryloxy (preferably phenoxy), polyalkylene oxide (preferably polyethylene oxide or polypropylene oxide), nitro, cyano, sulpho, halo, ureido, SO₂F, hydroxy, carboxy, ester; —NR¹R², —COR¹, —CONR¹R², —NHCOR¹, phosphato, sulphato, carboxyester, sulphone, and —SO₂NR¹R² wherein R¹ and R² are each independently H or alkyl (especially C₁₋₄-alkyl). It is more preferred that the optional substituents on X, A, D and E are selected from methyl, ethyl, propyl, butyl, phenyl, methoxy, ethoxy, butoxy, nitro, phenoxy, carboxy, phosphato, sulpho, sulphato, cyano, carboxyester, sulphone, sulphonamide, ureido, SO₂NR¹R² and NHCOR¹ groups.

In view of the preferences expressed hereinbefore it is especially preferred that the compound of Formula (1) is of Formula (3) or a salt thereof:

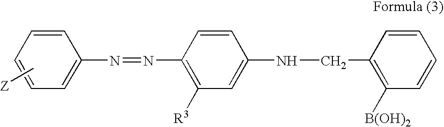

wherein:

Z is NO₂, CO₂H, OCH₃ or SO₃H; and

R³ is H, C₁₋₄-alkyl, OR⁴, NHCONH₂, NHCOR⁴, wherein R⁴ is H or alkyl.

Preferably Z is para with respect to the azo (—N═N—) group.

Preferred salts of the compounds described herein are alkali metal salts, especially lithium, sodium and potassium salts, ammonium and substituted ammonium salts and mixtures of the foregoing salts.

The compounds described herein may exist in tautomeric forms other than those shown in this specification. These tautomers are included within the scope of the present claims.

The compounds of Formula (1), (2), and (3) may be prepared as described in the co-pending application WO0112727.

In the method of the present invention the change in the spectral characteristics of the compound is preferably evaluated visually or, more preferably, using a spectrophotometer, fluorimeter or similar device. On binding to fluoride in the test sample the spectral properties of the compounds change, typically in terms of a change in fluorescent properties and/or a bathochromic or hypsochromic shift. There may also be an associated change in the intensity of light absorption.

As the compounds are ionisable their spectral properties can vary with the pH or their environment. Therefore the method is preferably performed at a constant pH preferably in the presence of a pH buffer. Suitable pH buffers are well known in the art, for example; phosphate, pyrophosphate, acetate, carbonate and citrate and mixed buffers such as citrate/phosphate.

The test sample preferably comprises water, it may also be a dental product, drink or foodstuff or derived from human or animal body fluid, a plant extract or a microbial fermentation. Usually such samples have been treated to remove coloured substances which could otherwise interfere with the method, for example blood may be centrifuged to remove red blood cells and urine may be decolorised with activated charcoal or some other substance which does not affect the quantity of fluoride in the test sample. Preferably the sample is industrial or domestic water or water intended for or extracted from an aquarium or other body of water in which aquatic animals live.

The compound of Formula (1), (2) and (3) used in the method is preferably in solution or attached to a water-insoluble carrier, preferably a cellulosic, glass or polymeric water-insoluble carrier. The carrier may be in any suitable form, for example as a flat support or beads.

The method of the invention preferably comprises the steps of:

a) contacting a test sample with a compound according to the present invention, wherein the compound is in solution or attached to a water-insoluble carrier;

b) evaluating the spectral characteristics of the compound when in contact with the test sample according to step a);

c) evaluating the extent of any change in the spectral characteristics determined in step b) compared to the spectral characteristics of the compound when the compound is not in contact with the test sample.

The method may be performed using an automated fluoride detection system, for example where the compound of the present invention and test sample are automatically introduced into a spectrophotometer or fluorimeter for evaluation of the spectral characteristics of the compound of the present invention after and optionally before contact with the test sample. The amount of fluoride may be measured by comparison with a series of predetermined standards or a calibration curve prepared by contacting the compound with a series of known concentrations of fluoride.

The spectral characteristics of a compound when the compound is not in contact with fluoride may be evaluated as part of the method normally before step a) or the spectral characteristics may already be known to the user e.g. from a colour chart or the general literature or a manual resulting in no need for this initial spectral characteristic to be measured as part of the method.

An advantage of this method is that the bathochromic or hypsochromic shift observed on reaction with fluoride is so large that the colour change may be clearly visible to the eye. Thus, the reaction of fluoride with a compound of Formula (1), (2) and (3) provides the basis of a rapid calorimetric test by visual comparison with a colour chart, in a similar manner to how pH is determined using litmus paper by comparison with a colour chart. This can be a particular advantage with domestic consumers and unskilled plant operators.

According to a second aspect of the invention there is provided a kit for detecting the presence and/or amount of fluoride which comprises:

a) a compound as described in the first aspect of the present invention, preferably in one or more pre-weighed portions or attached to a water-insoluble carrier;

b) instructions for determining the presence and/or amount of fluoride using the compound; and

c) optionally a pH buffer.

Preferably the instructions for determining the presence and/or amount of fluoride using component a) comprise the method according to the first aspect of the present invention. The invention is further illustrated by the following Examples.

### EXAMPLE 1

Compound 1 was prepared as in Example 1 in co-pending application WO0112727 which is herein incorporated by reference.

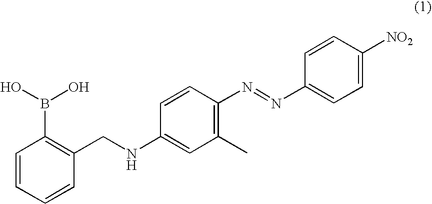

When a molar excess of any one of potassium chloride, bromide or iodide was added to compound (1) in solution in methanol there was an increase in the intensity of the main absorbance peak at 450 nm of around 20%, but no change in the absorbance maximum was observed. When a molar excess of potassium fluoride was added to compound (1) in solution in methanol there was an immediate visible change in the colour of the medium from orange (absorbance maximum 450 nm) to claret (absorbance maximum 563 nm).

### EXAMPLES 2-6

Example 1 may be repeated except that in place of compound (1) there is used compounds (2) to (6) described in Examples 2 to 6 on pages 7 to 10 of the co-pending international patent application WO0112727 which are incorporated herein by reference.

1. A method for detecting the presence and/or amount of fluoride in a test sample which comprises contacting the test sample with a compound of Formula (1) or a salt thereof and evaluating any change in the spectral characteristics of the compound:
X—[—(CₙH₂ₙ)—NH—(CₘH₂ₘ)—W—B(OH)₂]ₚ Formula (1)
wherein:
X is a chromophore or a fluorophore;
W is an optionally substituted aromatic ring;
n is 0, 1 or 2;
m is 1 or 2; and
p is 1 or 2.

2. A method according to claim 1 wherein the chromophore represented by X is optionally substituted azo anthraquinone, pyrroline, phthalocyanine, polymethine, aryl-carbonium, triphenodioxazine, diarylmethane, triarylmethane, anthraquinone, phthalocyanine, methine, polymethine, indoaniline, indophenol, stilbene, squarilium, aminoketone, xanthene, fluorone, acridene, quinolene, thiazole, azine, nigrosine, oxazine, thiazine, indigoid quinonioid, quinacridone, lactone, pyrroline, benzodifuranone, or indolene group or a combination of such groups.

3. A method according to claim 1 wherein the fluorophore represented by X is an optionally substituted naphthyl, anthryl, pyrenyl, stilbene and phenanthryl or a combination of such groups.

4. A method according to any one of claims 1 to 3 of Formula (2) or a salt thereof:
A—N═N—D—(CₙH₂ₙ)—NH—(CₘH₂ₘ)—E—B(OH)₂ Formula (2)
wherein:
A, D and E are each independently optionally substituted aromatic heterocyclic or homocyclic groups and m and n are as defined in claim 1.

5. A method according to claim 4 wherein A is optionally substituted phenyl and D and E are each independently optionally substituted phenylene.

6. A method according to any one of the preceding claims wherein the compound of Formula (1) is Formula (3) or a salt thereof: 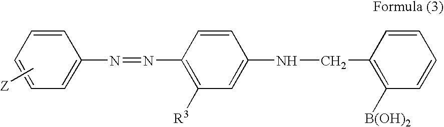
wherein:
Z is NO₂, CO₂H, OCH₃ or SO₃H; and
R³ is H or C₁₋₄-alkyl, OR⁴, NHCONH₂, NHCOR⁴, wherein R⁴ is H or alkyl.

7. A method according to any one of the preceding claims which is performed at a constant pH in the presence of a pH buffer.

8. A method according to any one of the preceding claims wherein the change in spectral characteristics is evaluated visually or by using a spectrophotometer or fluorimeter.

9. (Amended) A method according to claim 1 wherein the compound of formula (1), (2) and (3) is bound to a water-insoluble carrier:

10. (Amended) A method according to claim 1 which comprises the steps of:
a) contacting a test sample with a compound of Formula (1), (2) and (3) wherein the compound is in solution or attached to a water-insoluble carrier;
b) evaluating the spectral characteristics of the compound when in contact with the test sample according to step a);
c) evaluating the extent of any change in the spectral characteristics determined in step b) compared to the spectral characteristics of the compound when the compound is not in contact with the test sample.

11. (Amended) A kit for detecting the presence and/or amount of fluoride which comprises:
a) a compound as described in claim 1;
b) instructions for determining the presence and/or amount of fluoride using the compound; and
c) optionally a pH buffer.

12. (Amended) A kit according to claim 11 wherein the instructions for determining the presence and/or amount of fluoride using compound a) comprise a method for evaluating any change in the spectral characteristics of the compound when contacted with a test sample.
